(19) United States
(12) Patent Application Publication
(10) Pub. No.: US 2023/0306614 A1
(43) Pub. Date: Sep. 28, 2023

(54) SYSTEM AND METHOD FOR TRACKING SURGICAL KIT

(71) Applicant: Darvis Inc., San Francisco, CA (US)

(72) Inventors: Jan-Philipp Mohr, Hamburg (DE); Brian Earp, Merrimack, NH (US)

(21) Appl. No.: 17/706,260

(22) Filed: Mar. 28, 2022

(51) Int. Cl. G06T 7/20; A61B 90/94; A61B 50/30; H04N 5/247; H04N 5/232; G06T 7/11; G06T 7/00; G06V 20/52; G06T 7/70; G06K 7/14

(52) U.S. Cl. CPC G06T 7/20; A61B 90/94; A61B 50/30; H04N 5/247; H04N 5/23238; G06T 7/11; G06T 7/0012; G06V 20/52; G06T 7/70; G06K 7/1417; G06K 7/1413; G06T 2207/20132; G06T 2207/30004; G06V 2201/034

(57) ABSTRACT

Disclosed is a system arranged to track a surgical kit comprising a container and one or more surgical tools. The system comprises a unique identification tag for container and a unique identification tag for one or more surgical tools; and an image processor configured to receive at least one image of unique identification tag for container and/or unique identification tag for one or more surgical tools captured by at least one imaging arrangement and process at least one image to: identify unique identification tag for container and/or one or more surgical tools, determine identity of container and/or one or more surgical tools based on one or more parameters identified from unique identification tag, and track a location of container and/or one or more surgical tools, based on a location of at least one imaging arrangement that captured the image of container and/or one or more surgical tools.

### TECHNICAL FIELD

The present disclosure relates generally to tracking technology; and more specifically, to systems and methods for tracking a surgical kit comprising a container and one or more surgical tools.

### BACKGROUND

Generally, a sufficiently large facility that houses movable inventory items faces common problems of misplacement, inefficient use of the available space, messy use of the inventory, and so forth. This often results in a wastage of time in finding the inventory items at the right time. For instance, a hospital that uses ECG machines, medical equipment, beds and the like, must have an overview of the beds lying vacant, the number of ECG machines being used and so forth. Consequently, monitoring and tracking of such medical entities is a necessity.

One such movable inventory is a surgical kit that may be used at various levels (such as doctors, nurses, general duty assistants (for cleaning or transferring from thereof from one site to another, for example), and so on) at different time points in a medical facility (such as a hospital, a nursing home, and so forth). It will be appreciated that the surgical kit contains surgical tools which need to be cleaned regularly and/or replaced regularly. During a surgery, it is essential to have all the surgical tools present in the surgical kit. However due to regular cleaning/replacement, some surgical tools may be not present when they are needed, leading to a wastage of time whilst replacement items are found. Moreover, due to the number and precise nature of the surgical tools, it may not be immediately obvious which surgical tools are missing until the critical moment. Furthermore, replacement surgical tools may be purchased by spending an extra amount making the entire process cost inefficient.

Conventionally, monitoring and tracking may be a manual checking or maintaining digital logs. Notably, manually checking and keeping a count of each item is not feasible. Additionally, tracking all medical entities in a medical facility takes a significant amount of time and labor. Moreover, as manually checking of items is performed by employees, human errors can be introduced. Moreover, maintaining digital logs also involves human input and is time-consuming.

Recently, use of radio-frequency identification (RFID) technology and radio-frequency identification readers have been installed throughout such facilities to monitor and track the items such as surgical kits and surgical tools. However, these require each and every item, the surgical kit and well as each of the surgical tools associated therewith, to be tagged with a radio-frequency identification chip to be sufficiently close to the reader, which is rarely the case. Furthermore, another existing solution makes use of beacons (like Bluetooth low energy), mounted on the item and respective receivers throughout the medical facility. Consequently, the positions of the medical entities to be tracked are then located. A shortcoming of all this existing technology is that any attached beacons do not convey information about the state of the item, such as whether clean or not, date of expiry, and so forth.

Therefore, in light of the foregoing discussion, there exists a need to overcome the aforementioned drawbacks associated with conventional methods of tracking surgical kits within the facility.

### SUMMARY

The present disclosure seeks to provide a system arranged to track a surgical kit comprising a container and one or more surgical tools. The present disclosure also seeks to provide a method for tracking a surgical kit comprising a container and one or more surgical tools. An aim of the present disclosure is to provide a solution that overcomes at least partially the problems encountered in prior art.

In one aspect, the present disclosure provides a system arranged to track a surgical kit comprising a container and one or more surgical tools, the system comprising:
a unique identification tag for the container and a unique identification tag for the one or more surgical tools; and
an image processor configured to:
   receive at least one image of the unique identification tag for the container and/or the unique identification tag for the one or more surgical tools captured by at least one imaging arrangement, and
   process the at least one image to:
      identify the unique identification tag for the container and/or one or more surgical tools,
      determine the identity of the container and/or one or more surgical tools based on one or more parameters identified from the unique identification tag, and
      track a location of the container and/or one or more surgical tools, based on a location of the at least one imaging arrangement that captured the image of the container and/or one or more surgical tools.

In another aspect, the present disclosure provides a method for tracking a surgical kit comprising a container and one or more surgical tools, the method comprising:
utilising at least one imaging arrangement to capture at least one image of the container and/or one or more surgical tools, wherein the at least one image comprises a unique identification tag associated with the container and/or one or more surgical tools, and
utilising an image processor configured to receive the at least one image and process the at least one image to:
   identify the unique identification tag for the container and/or one or more surgical tools,
   determine the identity of the container and/or one or more surgical tools based on one or more parameters, and
   track a location of the container and/or one or more surgical tools, based on a location of an imaging arrangement that captured the image of the container and/or one or more surgical tools.

In yet another aspect, the present disclosure provides a computer program product comprising a non-transitory computer-readable storage medium having computer-readable instructions stored thereon, the computer-readable instructions being executable by a processing arrangement to execute the aforementioned method.

Embodiments of the present disclosure substantially eliminate or at least partially address the aforementioned problems in the prior art, and enable identifying the location of the surgical kit comprising the container and the one or more surgical tools associated therewith in real time. Additionally, the present disclosure further identifies the state, the class, and exact location of the container and the one or more surgical tools of the surgical kit within the facility using the unique identification tag associated with the container and the one or more surgical tools, respectively.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system arranged to track a surgical kit comprising a container and one or more surgical tools, in accordance with an embodiment of the present disclosure;

FIG. 2 is an illustration of an environment in which a system is arranged to track a surgical kit comprising a container and one or more surgical tools is implemented, in accordance with an embodiment of the present disclosure;

FIGS. 3A-3D illustrate steps of image processing using an image processor, in accordance with an embodiment of the present disclosure; and

FIG. 4 is an illustration of a flowchart depicting steps of a method for tracking a surgical kit comprising a container and one or more surgical tools, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, the present disclosure provides a system arranged to track a surgical kit comprising a container and one or more surgical tools, the system comprising:
a unique identification tag for the container and a unique identification tag for the one or more surgical tools; and
an image processor configured to:
   receive at least one image of the unique identification tag for the container and/or the unique identification tag for the one or more surgical tools captured by at least one imaging arrangement, and
   process the at least one image to:
      identify the unique identification tag for the container and/or one or more surgical tools,
      determine the identity of the container and/or one or more surgical tools based on one or more parameters identified from the unique identification tag, and
      track a location of the container and/or one or more surgical tools, based on a location of the at least one imaging arrangement that captured the image of the container and/or one or more surgical tools.

In another aspect, the present disclosure provides a method for tracking a surgical kit comprising a container and one or more surgical tools, the method comprising:
utilising at least one imaging arrangement to capture at least one image of the container and/or one or more surgical tools, wherein the at least one image comprises a unique identification tag associated with the container and/or one or more surgical tools; and
utilising an image processor configured to receive the at least one image and process the at least one image to:
   identify the unique identification tag for the container and/or one or more surgical tools,
   determine the identity of the container and/or one or more surgical tools based on one or more parameters, and
   track a location of the container and/or one or more surgical tools, based on a location of an imaging arrangement that captured the image of the container and/or one or more surgical tools.

In yet another aspect, the present disclosure provides a computer program product comprising a non-transitory computer-readable storage medium having computer-readable instructions stored thereon, the computer-readable instructions being executable by a processing arrangement to execute the aforementioned method.

The system and method of the present disclosure aim to provide tracking for a surgical kit comprising a container and one or more surgical tools.

In some implementations, the present disclosure can identify the location and one or more parameters (such as a type, a state and/or a class) associated with the container and/or one or more surgical tools in real time. In some implementations, the present disclosure can also provide for reconstructing the path of each tracked container and/or one or more surgical tools within a facility, such as a medical facility like a hospital. In this way, the system and method can enable the display of a dashboard of all the surgical kits and their spatial positions within the medical facility. In some implementations, the system and method can determine number of times a specific surgical kit has undergone a process to be measured. For an example, in a hospital, determining the number of times the surgical kit has been cleaned and reused in the past week.

Pursuant to embodiments of the present disclosure, the system and the method provided herein are for tracking a surgical kit comprising a container and one or more surgical tools. It will be appreciated that the surgical kit may be tracked in a facility such as a medical facility. Herein, the term medical facility refers to a place where healthcare is provided to people. The medical facility may typically include, but is not limited to, a hospital, a clinic, a nursing home, a maternity home, a medical school, a medical training institution, a health care facility, a physician's office, an infirmary, a dispensary, an ambulatory surgical centre, a sanatorium, or any other recognized institution or location where medical care is provided to any person. It will be appreciated that for delivering health care, the medical facility may require medical entities to be stored or managed from external sources temporarily as required. Herein, the term “surgical kit” refers to a collection of one or more surgical tools used by a trained health care professional. Moreover, the surgical kit includes: a container for containing the one or more surgical tools, such as scissors, scalpel, and so forth, therein. Herein, “tracking” refers to locating and identifying, the surgical kit present in the medical facility.

It will be appreciated that besides the surgical kit, a medical facility may have other trackable items (namely, medical entities) such as medical devices (for example a blood pressure monitoring device, an electrocardiogram machine, a pulse oximeter, and so forth), medical utility items (for example a bed, a wheelchair, a stretcher, crutches, and so forth), medical documents (for example a patient record, a prescription, a medical history, and so forth), and the like. Moreover, the medical entities may be movable assets and items in the medical facility that are required to be tracked routinely to ensure easy accessibility thereof when required.

The system comprises a unique identification tag for the container and a unique identification tag for the one or more surgical tools. Optionally, each of the one or more surgical tools may comprise a unique identification tag. Optionally, at least one of the one or more surgical tools may comprise a unique identification tag. Optionally, the one or more surgical tools may comprise more than one copy of a unique identification tag, for example one unique identification tag on the front and a copy of the unique identification tag on the back. Optionally, the container may comprise more than one copy of a unique identification tag, for example one unique identification tag on the front of the container and a copy of the unique identification tag on the back of the container.

Herein, the “unique identification tag” refers to an identification tag that may help in recognizing the container and/or one or more surgical tools. Additionally, the unique identification tag may be a series of numbers, alphabets, special characters, or any combination thereof. For each container and/or one or more surgical tools in the medical facility, the unique identification tag may be encoded in a visually distinctive and machine-readable pattern. Herein, visually distinctive and machine-readable pattern refers to a pattern that is easily recognizable, readable by a processor, such as an image processor, in an image captured by at least one imaging arrangement and that can be further processed by said processor. In some examples, the visually distinctive and machine-readable pattern may resemble a vehicle license plate, a quick response code (QR code), a colour code or any combination thereof.

Optionally, the unique identification tag is generated as a random machine-readable pattern. Herein, the unique identification tag is generated by the system randomly for the container and the one or more surgical tools associated with the surgical kit to be tracked within the medical facility. Additionally, the randomly generated unique identification tag may be printed and attached to at least one side of the container and the one or more surgical tools.

Optionally, the unique identification tag may contain one or more parameters (such as a type and/or a class, discussed below) associated with, the container and one or more surgical tools. For example, a type of container may be a box, a pouch, a tray.

The system comprises at least one imaging arrangement, positioned within the medical facility, to capture at least one image of the container and/or one or more surgical tools. The at least one imaging arrangement may be positioned in a manner that the unique identification tag is visible in at least one image captured by the at least one imaging arrangement. Herein, the at least one imaging arrangement may be installed within the medical facility in a way that substantially all potential areas for placing (namely storing) the surgical kit comprising the container and/or one or more surgical tools to be tracked are covered by the at least one imaging arrangement. Additionally, the at least one imaging arrangement may be installed and positioned in such a way that the visually encoded unique tag attached to at least one side of the container and/or one or more surgical tools is visible to the at least one imaging arrangement, irrespective of an orientation of the container and/or one or more surgical tools or even when the surgical kit is being moved. Beneficially, the at least one imaging arrangement is configured to track the container and the one or more surgical tools to locate them and identify which surgical tools are not present in the container.

Optionally, the at least one imaging arrangement includes a high optical zoom camera and a wide-angle camera. Notably, the high optical zoom camera combined with the wide-angle camera enables to efficiently see each individual unique identification tag for better tracking of the container and/or one or more surgical tools associated with the unique identification tag.

The system comprises an image processor. Herein, “image processor” refers to an image processing unit that can perform quantitative measurements of counts, length, duration, and thus can be used to analyse and process at least one image received from the at least one imaging arrangement. Additionally, the image processor may comprise software programs for creating and managing one or more processing tasks. For example, the image processor may perform noise reduction, object detection, extraction, and the like. Furthermore, the image processor is communicably coupled to the at least one imaging arrangement in the medical facility, e.g., via a network. Herein, a network may be a radio network, a local area network (LAN), wide area network (WAN), Personal Area Network (PAN), Wireless Local Area Network (WLAN), Campus Area Network (CAN), Metropolitan Area Network (MAN), storage-Area Network (SAN), and the likes.

The image processor, associated with the at least one imaging arrangement, is configured to receive at least one image of the unique identification tag for the container and/or the unique identification tag for the one or more surgical tools captured by at least one imaging arrangement and process the at least one image. Herein, an image or a set of consecutive images may be received from the at least one imaging arrangement via the network. The received image may contain the image of the container and/or one or more surgical tools to be tracked. Additionally, the side of the container and/or one or more surgical tools comprising the unique identification tag may be visible in the said received image. Herein, the image processor may process the received image to recognize the presence and position of the container and/or one or more surgical tools within the image, and specifically, the unique identification tag of the container and/or one or more surgical tools.

Optionally, the image processor is configured to employ at least one of: computer vision, neural networks, image processing algorithms for processing the at least one image. Optionally, the image processor may employ neural networks for processing of the at least one image and the image processor may be trained using several training data sets of real images, synthetic images or a combination thereof for identification of one or more parameters associated with the container and/or one or more surgical tools. For example, the images used in the training data sets may be similar to the images of the container and/or one or more surgical tools to be stored in the medical facility. Different angles, different backgrounds and variable distances of the container and/or one or more surgical tools may be used to train the image processor. Additionally, algorithms such as You Only Look Once (YOLO) algorithm, MM algorithm may be used for object detection in the neural network of the image processor. Herein, “object” refers to the container and/or one or more surgical tools in the image.

Moreover, the image processor is configured to process the at least one image to identify the unique identification tag for the container and/or one or more surgical tools by correlating one or more parameters associated with the container and/or one or more surgical tools. It will be appreciated that the container and/or one or more surgical tools has specific one or more parameters which help identification thereof.

Optionally, the one or more parameters include at least one of: a type, a class, a state of the container and/or one or more surgical tools. The image processor may detect the state of the container and/or one or more surgical tools, the class, the type or any combination thereof. Herein, “state” refers to the actual condition or the situation of a container and/or one or more surgical tools. Optionally, the state may relate to a new entity, a cleaned entity, a used entity, a free state, an in-use state, an occupied state, a worn-out state, and so forth. In this regard, for an example, in a hospital, the state of a hospital bed may be empty or occupied, and the state of a surgical tool may be cleaned for reuse or a new purchase. Furthermore, the term “type” and “class” may refer to a predefined category of the container and/or one or more surgical tools. Such predefined categories may be stored with the image processor. In an example, the type of a container and/or one or more surgical tools may be an invasive device or a non-invasive device. In this regard, the class of a container and/or one or more surgical tools may be injection or forceps, a clamp, a scissor, and so forth.

Furthermore, the image processor is configured to determine the identity of the container and/or one or more surgical tools based on one or more parameters identified from the unique identification tag In some examples, the image processor may be trained to locate the position and identify the unique identification tag in the cropped image. Herein, the image processor identifies and locates the unique identification code in the cropped image. In some examples, the cropped image may focus on the container and/or one or more surgical tools alone and discard the noise present in the image. As such, the cropped image may be left with less data for the image processing unit to process. In this way, the cropped image can make it easier for the image processor to locate and identify the unique identification tag.

Additionally, the image processor may be configured to determine the identity of the container and/or one or more surgical tools from the unique identification tag thereof. Herein, the image processor may be trained to decode the identified unique identification tag in the cropped image. In some examples, the decoded data from the unique identification tag of the cropped image may be extracted. In some examples, the extracted data may be compared with a database to determine the type of the container and/or one or more surgical tools.

Optionally, the image processor is configured to crop the at least one image to bounding box coordinates of the container and/or one or more surgical tools, prior to identifying the unique identification tag associated with the container and the one or more surgical tools. Herein, bounding box coordinates refers to a box shaped outline around the container and the one or more surgical tools in the image. The box shaped outline focuses on the position and presence of the container and the one or more surgical tools in the image. In some examples, the image processor may crop the image to the bounding box coordinates. In some examples, cropping the image may remove noise elements present in the image. Herein, noise element refers to unnecessary elements present in the background of the image and around the image. In some examples, the medical entity within the boundary box may be enlarged and/or filtered to enhance readability of the unique identification tag. In some examples, the image processor may identify the unique identification tag and decodes the same using at least one algorithm.

Furthermore, the image processor is configured to track a location of the container and the one or more surgical tools, based on a location of the at least one imaging arrangement that captured the image of the container and the one or more surgical tools. In this regard, the position of the imaging arrangement that captured the said image of the container and the one or more surgical tools is obtained by the database comprising the details of the at least one imaging arrangement positioned within the facility. Optionally, the defined position of the imaging arrangement within the medical facility may be associated with the location of the container and the one or more surgical tools in the processed image.

Optionally, the image processor is communicably coupled with a database. The term “database” as used herein relates to an organized body of digital information regardless of the manner in which the data or the organized body thereof is represented. Optionally, the database may be hardware, software, firmware and/or any combination thereof. Furthermore, the database may comprise software programs for creating and managing one or more databases. Optionally, the database may be operable to support relational operations, regardless of whether it enforces strict adherence to the relational model, as understood by those of ordinary skill in the art. Additionally, the database may be populated by data elements. Furthermore, the data elements may include data records, bits of data, cells, which are used interchangeably herein and are all intended to mean information stored in cells of a database.

The database is configured to store a data corresponding to the one or more parameters associated with the container and/or one or more surgical tools; the unique identification tag associated with the container and/or one or more surgical tools; and the tracked location of the container and/or one or more surgical tools. The image processor may be configured to employ the database for determining the identity of a given container and/or one or more surgical tools associated with a given unique identification tag and track a location of the container and/or one or more surgical tools based on the location of the imaging arrangement that captured the image of the container and/or one or more surgical tools. Herein, the database records all the information of each container and/or one or more surgical tools at the time of entry into to the medical facility. Additionally, the unique identification tag generated by the system may also be recorded corresponding to each container and/or one or more surgical tools. The information stored in the database may contain the information relating to container and/or one or more surgical tools, such as an ideal position thereof, a changed location thereof, one or more parameters associated with the container and/or one or more surgical tools, at least one imaging arrangement positioned within the medical facility to capture at least one image of the container and/or one or more surgical tools, availability, date of entry of the container and/or one or more surgical tools in the medical facility, and so forth. In some examples, the unique identification tag received from the image of at least one imaging arrangement in the medical facility may be compared with the stored information in the database. In some examples, the information matching the unique identification tag may be extracted from the database and the associated container and/or one or more surgical tools type may be determined.

In some embodiments, the image processor may be further configured to employ a filter algorithm for collecting data relating to a given unique identification tag from a plurality of consecutive image frames and extract a legible image of the given unique identification tag therefrom. Herein, a “legible image” refers to a clear enough image to be read easily by the image processor. In some cases, the unique identification tag in the image may be in part or entirely illegible due to a bad read angle from the at least one of the cameras, bad lightning, obscuring through people or other objects, or any other factor. A filter algorithm may be employed to construct at least one legible image frame with the unique identification tag from a plurality of consecutive image frames. Herein, the “consecutive image frames” refer to successive images taken by at least one of the at least one imaging arrangement installed in the medical facility. In some examples, the filter algorithm employed may be heuristic, Kalman or any other type to extract the unique identification tag from the plurality of consecutive frames. In some examples, the extracted data of the unique identification tag from the filtered image frame may be compared with same class objects in the database. In this regard, in a case where a matching unique identification tag is identified, it can be considered valid. In some examples, the distance of the unique identification tag may be calculated in relation to all unique identification tags of the same object class in the database and the closest distance can be considered to be the valid unique identification tag. In some examples, in a case where the extracted data of the unique identification tag from the filtered image frame does not match with the database, the image frame may be discarded. In some examples, a new image may be considered again.

Optionally, the image processor is configured to use a homographic matrix for calculating, based on the unique identification tag of the container and/or one or more surgical tools, a spatial position of the container and/or one or more surgical tools. For example, the homographic matrix may be used on the valid image with valid unique identification tag to calculate the spatial position of the container and/or one or more surgical tools. Herein, “spatial position” refers to the position or the location of the container and/or one or more surgical tools being tracked within the medical facility. In some examples, the bounding box position of the container and/or one or more surgical tools in the image, the imaging arrangement that detected the medical entity, the time stamp of the detection and the detected state of the container and/or one or more surgical tools may be sent to the database for storage. In this regard, the database may be equipped with the time-stamp series data and the spatial positions associated with the container and/or one or more surgical tools. Moreover, the data can keep updating as soon as a container and/or one or more surgical tools within the medical facility is moved. In some examples, the data may also be updated after a fixed regular interval or at the time of need.

Optionally, the identified unique identification tag and hence the container and/or one or more surgical tools may be positioned within a two-dimensional map by applying a homographic projection to the centre image coordinates of the unique identification tag. For example, the homographic projection may be applied using a homographic matrix derived from defining four points within the image and four corresponding points on a ground map. In some examples, this method can yield a precise position for a known mounting height of the unique identification tag. In some examples, the ground plane of an image may be determined, and the position of unique identification tag may be calculated by the intersection of a vertical line from its centre and the ground plane. The resulting image coordinates may be projected onto a ground map using the homographic projection.

In some embodiments, the unique identification tag of the container and/or one or more surgical tools may only need to be identified once using the at least one imaging arrangement within a medical facility. Subsequently, the identified unique identification tag may be assigned to the container and/or one or more surgical tools and may be tracked throughout the medical facility. In some embodiments, the information of the unique identification tag may be updated in the database in the event that the same unique identification tag is re-identified by a second camera image at a later stage.

Optionally, the image processor is further configured to generate a notification in case of a change in one or more parameters associated with the container and/or one or more surgical tools; or a presence or an absence of the container and/or one or more surgical tools at a desired location. The term “notification” as used herein refers to an alert corresponding to a change in one or more parameters associated with the container and/or one or more surgical tools or the desired location of finding it. It will be appreciated that an alert algorithm may be employed to generate notifications in case of: a change in one or more parameters associated with the container and/or one or more surgical tools or a presence or an absence of the container and/or one or more surgical tools.

Optionally, the notification is generated at an authorized user of the system. The authorized user of the system may be any professional employed by the medical facility and is responsible for the availability of the container and/or one or more surgical tools at a defined location. Optionally, the authorized user may be a healthcare professional, a general duty assistant, operations professionals, and so forth. The authorised user after receiving the notification is required to perform a desired action, such as to restore the changed one or more parameters or the location of the container and/or one or more surgical tools to circumvent the emergency situation. Optionally, the notifications may be generated to the authorised user via a software application on the graphical user interface of the system or an associated user device coupled to the system.

It will be appreciated that it is possible to reconstruct the path of each of the tracked surgical kit comprising the container and the one or more surgical tools within the medical facility. Additionally, locating the last known position of the surgical kit is possible in-real time. In this regard, a dashboard with all the surgical kits and their spatial positions may be displayed. In some examples, one or more actions may be performed in real time based on the state and location of a specific surgical kit within the medical facility. For an example, in a hospital, if a surgical kit “A” is cleaned in room “B”, then bring back the cleaned surgical kit “A” to nursing station “C”. Herein, the medical entity is the surgical kit “A”, the state of the surgical kit is “cleaned” and the action performed is bringing back the surgical kit to its desired location “C”.

The present disclosure also relates to the method as described above. Various embodiments and variants disclosed above apply mutatis mutandis to the method.

Optionally, the image processor is configured to employ at least one of: computer vision, neural networks, image processing algorithms for processing the at least one image.

Optionally, the method comprises utilising the image processor to crop the at least one image to bounding box coordinates of the container and/or one or more surgical tools, prior to identifying the unique identification tags of the container and/or one or more surgical tools.

Optionally, the method comprises utilising the image processor to use a homographic matrix for calculating, based on the unique identification tag of the surgical kit, a spatial position of the container and/or one or more surgical tools.

Optionally, the method further comprises utilising a database, communicably coupled to the image processor, for storing a data corresponding to:
the one or more parameters associated with the container and/or one or more surgical tools;
the unique identification tag associated with the container and/or one or more surgical tools; and
the tracked location of the container and/or one or more surgical tools.

Optionally, the one or more parameters include at least one of: a type, a class, a state of the container and/or one or more surgical tools.

Optionally, the at least one imaging arrangement includes a high optical zoom camera and a wide-angle camera.

Optionally, the method further comprises configuring the image processor to generate a notification in case of:
a change in one or more parameters associated with the container and/or one or more surgical tools; or
a presence or an absence of the container and/or one or more surgical tools.

Optionally, the unique identification tag is at least one of a bar code, a QR code, or a random machine-readable pattern.

The present disclosure also relates to the computer program product as described above. Various embodiments and variants disclosed above apply mutatis mutandis to the computer program product.

The computer program product comprising a non-transitory computer-readable storage medium having computer-readable instructions stored thereon, the computer-readable instructions being executable by a processing arrangement to execute the aforementioned method.

### DETAILED DESCRIPTION OF THE DRAWINGS

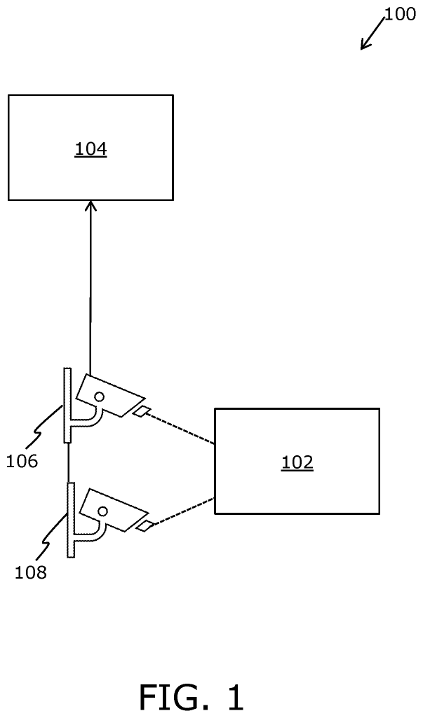

Referring to FIG. 1, there is shown a block diagram illustrating system 100 arranged to track a surgical kit comprising a container and one or more surgical tools, in accordance with an embodiment of the present disclosure. The system 100 comprises a unique identification tag (not shown) for the container 102 and a unique identification tag for the one or more surgical tools (not shown), and an image processor 104. The image processor 104 is configured to receive at least one image of the unique identification tag for the container 102 and/or the unique identification tag for the one or more surgical tools captured by at least one imaging arrangement 106, 108 and process the at least one image. Moreover, the image processor 104 is configured to process the at least one image to identify the unique identification tag for the container 102 and/or one or more surgical tools, determine the identity of the container 102 and/or one or more surgical tools based on one or more parameters identified from the unique identification tag, and track a location of the container 102 and/or one or more surgical tools, based on a location of the at least one imaging arrangement that captured the image of the container 102 and/or one or more surgical tools.

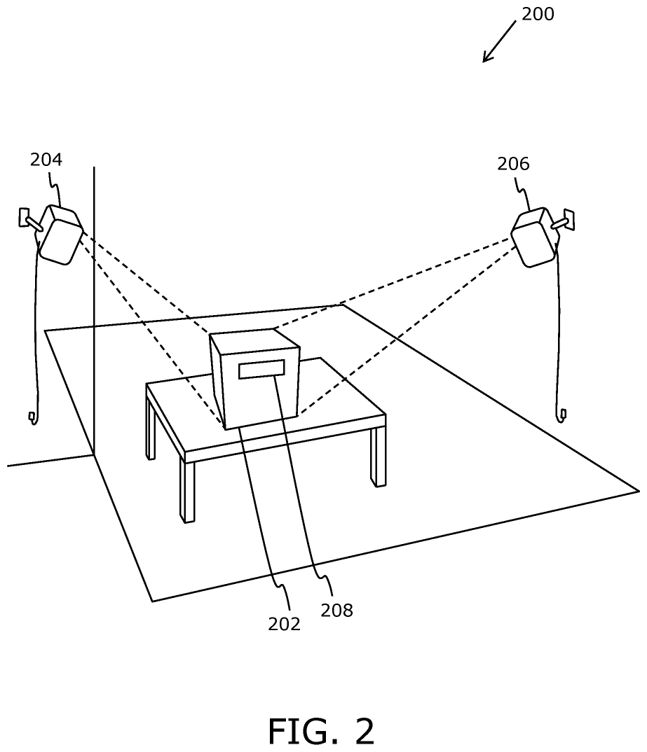

Referring to FIG. 2, illustrated is an environment in which a system 200 is arranged to track a surgical kit comprising a container 202 and one or more surgical tools (not shown) is implemented, in accordance with an embodiment of the present disclosure. As shown, the system 200 comprises at least one imaging arrangement, such as imaging arrangements 204, 206 configured to captures at least one image of a unique identification tag 208 for the container 202. Hereinafter, the at least one image is sent to an image processor for further processing of the at least one images.

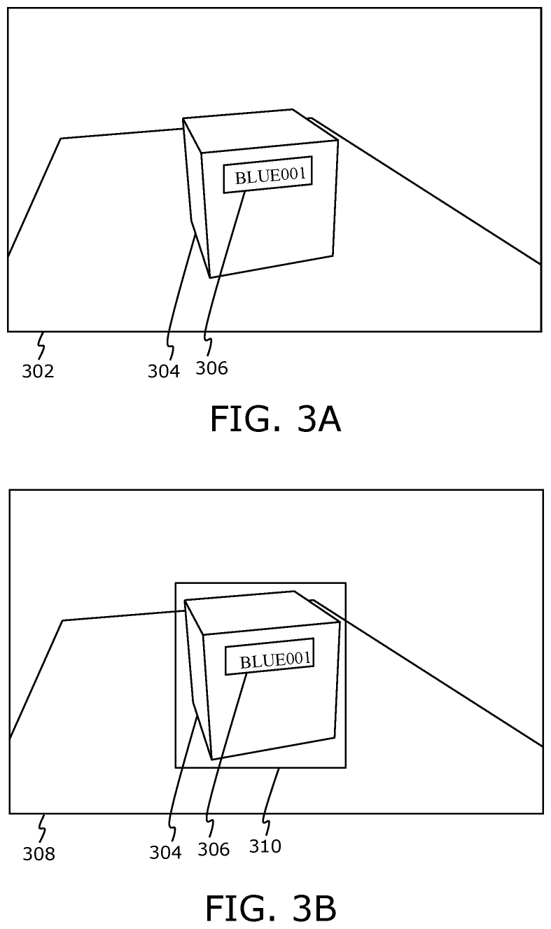

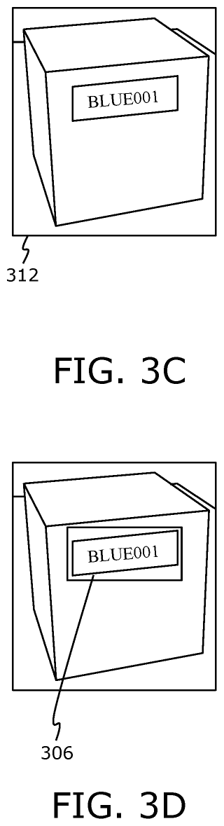

Referring to FIGS. 3A-3D, illustrated are steps of image processing by an image processor, in accordance with an embodiment of the present disclosure. FIG. 3A shows an image 302 of a container 304 comprising a unique identification tag 306 as captured by at least one imaging arrangement (not shown) which is associated with an image processor. FIG. 3B shows an image 308 wherein the container 304 comprising the unique identification tag 306 has a bounding box 310 therearound. In FIG. 3C, the image 312 is cropped according to the bounding box 310. In FIG. 3D, the image processor identifies the unique identification tag 306 in the image 312.

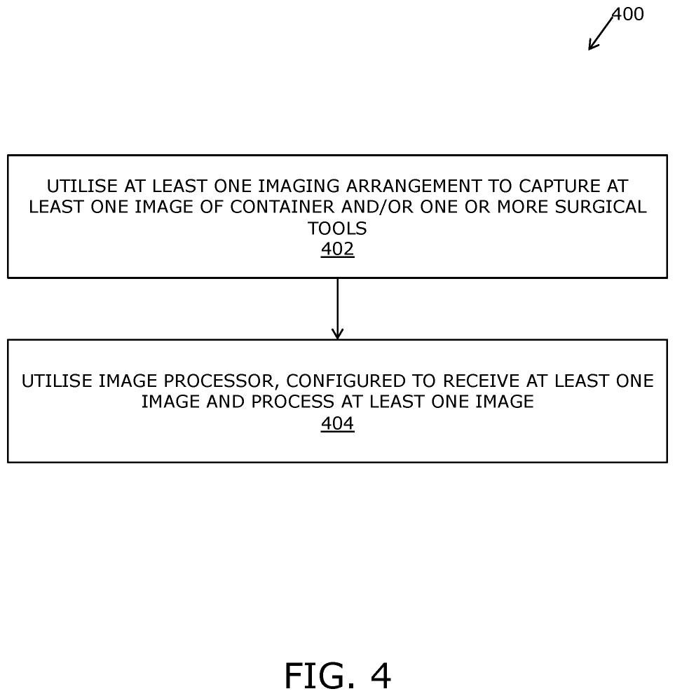

Referring to FIG. 4, illustrated is a flowchart 400 depicting steps of a method for tracking a surgical kit comprising a container and one or more surgical tools, in accordance with an embodiment of the present disclosure. At step 402, at least one imaging arrangement is utilised to capture at least one image of the container and/or one or more surgical tools, wherein the at least one image comprises a unique identification tag associated with the container and/or one or more surgical tools. At step 404, an image processor is utilised and is configured to receive the at least one image and process the at least one image to: identify the unique identification tag for the container and/or one or more surgical tools, determine the identity of the container and/or one or more surgical tools based on one or more parameters, and track a location of the container and/or one or more surgical tools, based on a location of an imaging arrangement that captured the image of the container and/or one or more surgical tools.

The steps 402 and 404 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1.-19. (canceled)

20. A system arranged to track a surgical kit comprising a container and one or more surgical tools, the system comprising:
a unique identification tag for the container and a unique identification tag for the one or more surgical tools; and
an image processor configured to:
receive at least one image of the unique identification tag for the container and/or the unique identification tag for the one or more surgical tools captured by at least one imaging arrangement, and
process the at least one image to:
identify the unique identification tag for the container and/or one or more surgical tools,
determine the identity of the container and/or one or more surgical tools based on one or more parameters identified from the unique identification tag, and
track a location of the container and/or one or more surgical tools, based on a location of the at least one imaging arrangement that captured the image of the container and/or one or more surgical tools.

21. A system of claim 20, wherein the image processor is configured to employ at least one of: computer vision, neural networks, image processing algorithms for processing the at least one image.

22. A system of claim 20, wherein the image processor is configured to crop the at least one image to bounding box coordinates of the container and/or one or more surgical tools, prior to identifying the unique identification tag of the container and/or one or more surgical tools.

23. A system of claim 20, wherein the image processor is configured to use a homographic matrix for calculating, based on the unique identification tag of the container and/or one or more surgical tools, a spatial position of the container and/or one or more surgical tools.

24. A system of claim 20, wherein the image processor is communicably coupled with a database, wherein the database is configured to store data corresponding to:
the one or more parameters associated with the container and/or one or more surgical tools;
the unique identification tag associated with the container and/or one or more surgical tools; and
the tracked location of the container and/or one or more surgical tools.

25. A system of claim 20, wherein the one or more parameters include at least one of: a type, a class, a state of the container and/or one or more surgical tools.

26. A system of claim 20, wherein the at least one imaging arrangement includes a high optical zoom camera and a wide-angle camera.

27. A system of claim 20, wherein the image processor is further configured to generate a notification in case of:
a change in one or more parameters associated with the container and/or one or more surgical tools; or
a presence or an absence of the container and/or one or more surgical tools.

28. A system of claim 20, wherein the unique identification tag is at least one of a bar code, a Quick Response code, or a random machine-readable pattern.

29. A method for tracking a surgical kit comprising a container and one or more surgical tools, the method comprising:
utilising at least one imaging arrangement to capture at least one image of the container and/or one or more surgical tools, wherein the at least one image comprises a unique identification tag associated with the container and/or one or more surgical tools; and
utilising an image processor configured to receive the at least one image and process the at least one image to:
identify the unique identification tag for the container and/or one or more surgical tools,
determine the identity of the container and/or one or more surgical tools based on one or more parameters, and
track a location of the container and/or one or more surgical tools, based on a location of an imaging arrangement that captured the image of the container and/or one or more surgical tools.

30. A method of claim 29, wherein the image processor is configured to employ at least one of: computer vision, neural networks, image processing algorithms for processing the at least one image.

31. A method of claim 29, wherein the method comprises utilising the image processor to crop the at least one image to bounding box coordinates of the container and/or one or more surgical tools, prior to identifying the unique identification tags of the container and/or one or more surgical tools.

32. A method of claim 29, wherein the method comprises utilising the image processor to use a homographic matrix for calculating, based on the unique identification tag of the surgical kit, a spatial position of the container and/or one or more surgical tools.

33. A method of claim 29, wherein the method further comprises utilising a database, communicably coupled to the image processor, for storing a data corresponding to:
the one or more parameters associated with the container and/or one or more surgical tools;
the unique identification tag associated with the container and/or one or more surgical tools; and
the tracked location of the container and/or one or more surgical tools.

34. A method of claim 29, wherein the one or more parameters include at least one of: a type, a class, a state of the container and/or one or more surgical tools.

35. A method of claim 29, wherein the at least one imaging arrangement includes a high optical zoom camera and a wide-angle camera.

36. A method of claim 29, wherein the method further comprises configuring the image processor to generate a notification in case of:
a change in one or more parameters associated with the container and/or one or more surgical tools; or
a presence or an absence of the container and/or one or more surgical tools.

37. A method of claim 29, wherein the unique identification tag is at least one of a bar code, a QR code, or a random machine-readable pattern.

38. A computer program product comprising a non-transitory computer-readable storage medium having computer-readable instructions stored thereon, the computer-readable instructions being executable by a processing arrangement to execute a method of claim 29.
